(19) United States
(12) Patent Application Publication
(10) Pub. No.: US 2018/0214331 A1
(43) Pub. Date: Aug. 2, 2018

(54) SURGICAL FLOOR MAT FOR COLLECTING FLUIDS

(71) Applicant: Peter Borden, Torrance, CA (US)

(72) Inventor: Peter Borden, Torrance, CA (US)

(21) Appl. No.: 15/882,232

(22) Filed: Jan. 29, 2018

Related U.S. Application Data
(60) Provisional application No. 62/452,274, filed on Jan. 30, 2017.

(51) Int. Cl. A61G 13/10; A47L 7/00; A61M 1/00

(52) U.S. Cl. CPC A61G 13/102; A61M 1/008; A47L 7/0004

(57) ABSTRACT

A non-disposable, protective surgical floor mat system that is made of a bodily fluid-impervious material, has raised borders, and drainage slots adapted to contain spillage of surgical fluids, and incorporates a disposable suction device that is adapted to drain, permit suction of or otherwise permit removal of the contained fluids while the surgical procedure is being performed or afterward.

### FIELD OF INVENTION

The invention relates generally to floor mats used in medical surgical procedures and adapted for containing spillage of fluids that escape the body or are otherwise used during surgery.

### BACKGROUND

During certain surgical procedures, fluids such as saline, blood, and urine can escape the body and flow to the floor causing an expanding mess of fluids that are typically contained by operative personnel by placing towels or blankets around the area of spillage to avoid further expansion of the contaminated floor. This current method of containment is not truly effective and results in higher hospital or surgical center costs of soiled linens that may require replacement or laundry fees. Currently, there are disposable commercial mats available that absorb the leaking fluids and even some that have internal suction devise, however, many of these are minimally effective in containing leakage of fluids and cause increasing costs due to their recommended one time use per surgery.

### SUMMARY OF INVENTION

The surgical mat according to the present disclosure overcomes the drawbacks of known surgical mats by providing the capability to remove fluids resulting from surgical procedures using standard operating room suction tubing.

Embodiments, examples, features, aspects, and advantages of the present disclosure will become better understood with regard to the following description, appended claims and accompanying drawings.

The invention relates to a non-disposable, protective floor mat system that contains the spillage of surgical fluids and incorporates a disposable suction device that allows for elimination of the contained fluids while the surgical procedure is being performed.

A preferred embodiment floor mat is composed of a plastic, plastic like, or rubber material that is impervious to fluids and is intended to repel the fluids within the impervious raised borders surrounding the periphery of the floor mat. The mat is designed to be stood on by the surgeon and or assistants while the procedure is performed. Following and during the procedure, the fluids are removed from the mat by the additional mobile disposable suction device for faster clean up and turn over time between surgical procedures. The mat material allows for cleaning with standard bacteriostatic cleaning solutions to minimize cross contamination between surgical procedures.

Embodiments of the mat can be square, rectangular, triangular, or circular shaped with appropriate raised borders depending on the shape of the mat.

A preferred embodiment suction device is made of a disposable plastic material that sits on the floor of the mat and has small slots on the undersurface of the device that are continuous with a central suction tubing hook up attachment that allows for standard operating room suction tubing to be connected. The suction device allows for manual movement within the borders of the mat for placement in the desired location at the preference of the surgeon or nursing personnel.

Indications for use of the floor mat system can be expanded to other environments other than the operating room of a hospital or surgical center for the purpose of containing and eliminating spillage of fluids.

### BRIEF DESCRIPTION OF THE DRAWINGS

The foregoing aspects and the attendant aspects of the present disclosure will become more readily appreciated by reference to the following detailed description, when taken in conjunction with the accompanying drawings, wherein:

FIG. 1a is a top perspective view of a preferred embodiment surgical floor mat;

FIG. 1b is a top perspective view of a raised border surrounding the edge of the FIG. 1a embodiment mat

FIG. 2a is a top view of a preferred suction device of the FIG. 1a embodiment; and,

FIG. 2b is a bottom view of the FIG. 2a suction device

FIG. 2c is a side cross-section view of the FIG. 2a suction device;

FIG. 3a is a top perspective view of preferred embodiment floor mat system, including the FIG. 1a embodiment mat and the FIG. 2a suction device embodiment placed within the floor mat and connected to suction tubing;

FIG. 3b is top perspective view of the suction device of the FIG. 3a system embodiment; and,

FIG. 3c is a top perspective view of the suction tubing of the FIG. 3a system embodiment.

Reference symbols or names are used in the Figures to indicate certain components, aspects or features shown therein. Reference symbols common to more than one Figure indicate like components, aspects or features shown therein.

### DETAILED DESCRIPTION OF INVENTION

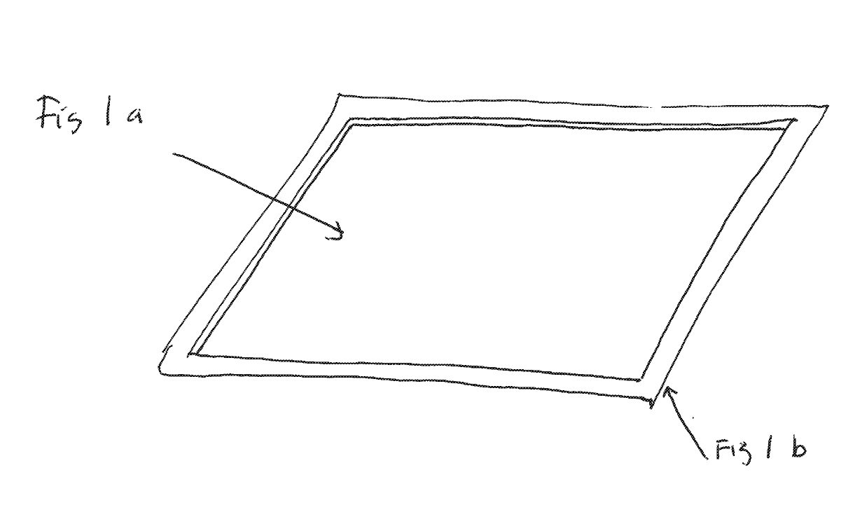

In accordance with embodiments described herein FIGS. 1-3 show a preferred embodiment surgical floor mat system for collecting and removing fluids that are associated with surgical procedures.

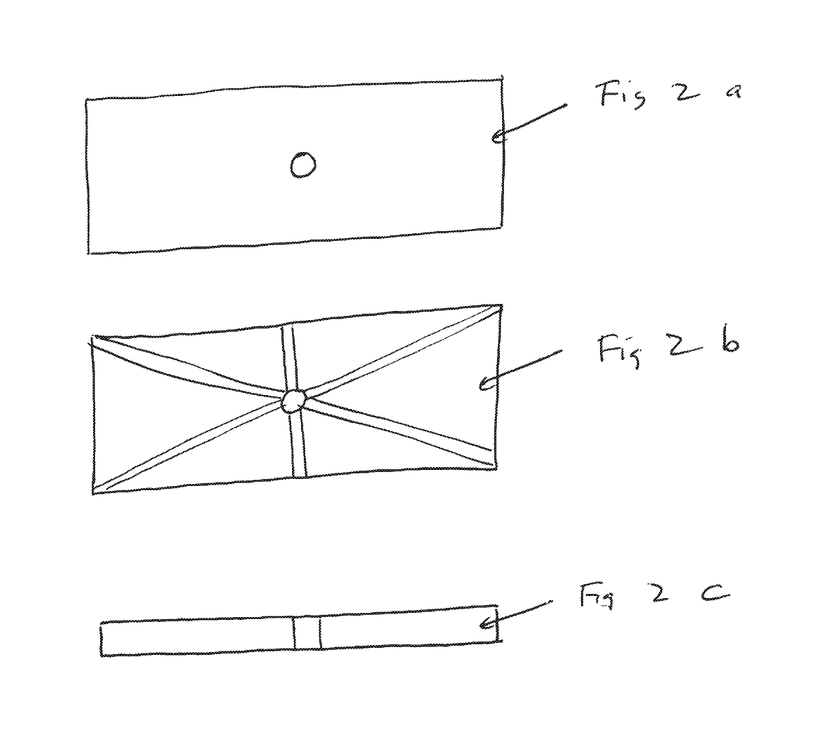

Referring to FIGS. 1a and 1b, a preferred embodiment floor mat is composed of a plastic, plastic like, or rubber material that is impervious to fluids and is intended to repel the fluids within the impervious raised borders surrounding the periphery of the floor mat. The mat is designed to be stood on by the surgeon and or assistants while the procedure is performed. Referring to FIGS. 2a-2c, following and during the surgical procedure, the fluids are removed from the mat by the illustrated mobile disposable suction device for faster clean up and turn over time between surgical procedures. The mat material allows for cleaning with standard bacteriostatic cleaning solutions to minimize cross contamination between surgical procedures.

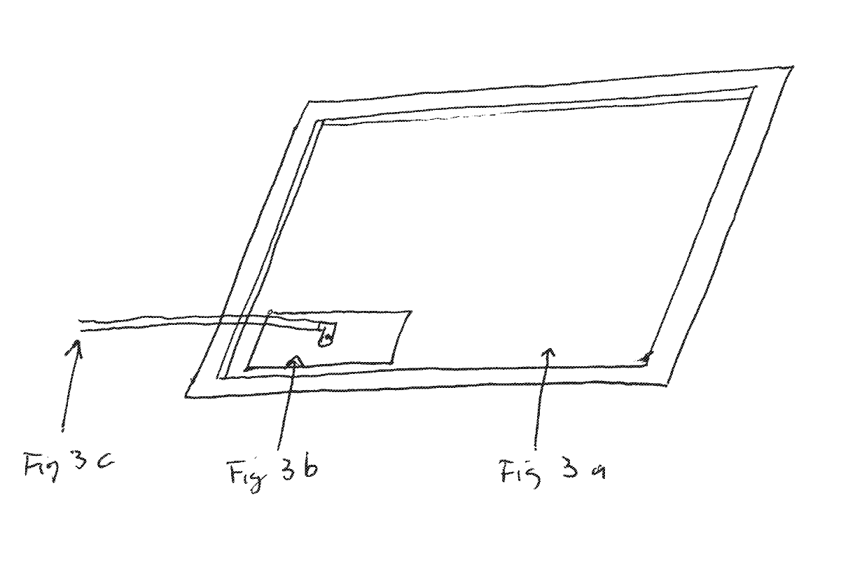

Referring to FIGS. 3a-3c, a preferred location of the suction device is shown, together with connected suction tubing.

Although specific embodiments of the disclosure have been described, various modifications, alterations, alternative constructions, and equivalents are also encompassed within the scope of invention as set forth in the claims.

The specification and drawings are, accordingly, to be regarded in an illustrative rather than a restrictive sense. It will, however, be evident that additions, subtractions, deletions, and other modifications and changes may be made thereunto without departing from the broader spirit and scope of invention as set forth in the claims.

What is claimed is:

1. A surgical floor mat system for collecting fluid comprising:
a mat impervious to fluids associated with surgical procedures such as saline and bodily fluids, having a top side, an under side, predetermined length, a predetermined width, a predetermined thickness, and having a raised border extending around the periphery of the mat;
a hole in the mat, the hole extending though the thickness of the mat;
a plurality of slots positioned on the underside of the mat, each slot extending from a predetermined slot end to the hole in the mat; and,
a fitting positioned in the hole in the mate, and adapted for connection to conventional operating room suction tubing.
